(19) United States
(12) Patent Application Publication
(10) Pub. No.: US 2026/0325746 A1
(43) Pub. Date: Oct. 22, 2026

(54) Methods and apparatus for subcutaneous thyroid hormone delivery

(71) Applicant: Brian Haney, Las Vegas, NV (US)

(72) Inventor: Brian Haney, Las Vegas, NV (US)

(73) Assignee: Brian Haney, Las Vegas, NV (US)

(21) Appl. No.: 19/698,986

(22) Filed: Jun. 5, 2026

Related U.S. Application Data
(63) Continuation-in-part of application No. 17/734,024, filed on Apr. 30, 2022.

(51) Int. Cl. A61M 5/142; A61M 5/172; G16H 20/17; G16H 40/63

(52) U.S. Cl. CPC A61M 5/14276; G16H 20/17; G16H 40/63; A61M 2005/1726; A61M 2205/3317; A61M 2230/20

(57) ABSTRACT

A method and apparatus for thyroid hormone control using a synthetic module containing thyroid hormone and an intelligent valve control device. In embodiments, the invention consists of an iterative process with three steps. First, blood is measured to detect thyroid hormone concentration. Second, the measured concentration is compared to a target thyroid hormone concentration. Third, the intelligent valve control device optimizes thyroid hormone delivery, correcting for differences between the measured concentration and the target concentration.

### BACKGROUND TO THE INVENTION

The field of the invention rests at the intersection of two broader fields, artificial intelligence and endocrinology. Endocrinology is a medical branch concerned with endocrine glands and hormones, including the thyroid and thyroid hormones. Artificial intelligence refers to computational processes mirroring the human mind's thoughtful deliberation, decision making, and action-oriented behavior. Specifically, the field of the invention is artificial intelligence for drug delivery to patients with hypothyroidism, the most common thyroid disease in the United States.

Hypothyroidism is an endocrine disease affecting an estimated 9,900,000 people in the United States, 37,070,000 in Europe, and 104,000,000 people globally. Conventional methods for curing the disease fail for various reasons leading to ongoing patient symptoms including weight gain, depression, and anxiety. Further, medical reports suggest a common trend is that more than half of patients with hypothyroidism are undiagnosed and untreated. Moreover, there are several hypothyroidism types. For example, Congenital Hypothyroidism (CH) is defined as thyroid hormone deficiency present at birth. Another example is Hashimoto's Disease, a hypothyroid condition in which the immune system attacks the human thyroid gland preventing adequate Thyroxine (T₄) production.

The thyroid is a bilobal gland located at the base of the neck in front of the windpipe. The thyroid's functionality, essentially synthesizing thyroid hormone, is meticulously modeled through a feedback loop. The pituitary gland and thyroid communicate instructions for controlling hormone production and stabilization. The system works on an iterative loop where cells in the pituitary gland determine the body's normal hormonal range, known as the set point. In other words, the thyroid produces hormones, which are secreted into the blood and then carried to every tissue in the body. As such, the Thyroid produces a hormonal variety governing the human body's metabolism.

Thyroid hormone synthesis is a three-step process. First, the hypothalamus produces Thyroid Releasing Hormone (TRS), stimulating the pituitary gland to release Thyroid Stimulating Hormone (TSH), Thyrotropin, which in turn activates the thyroid gland. Second, the thyroid gland excretes Thyroxine (T₄) to the bloodstream. Third, T₄ converts to Triiodothyronine (T₃) through deiodination in peripheral tissues. This synthesis is critical for metabolic control in the human body.

Thyroxine (T₄) converts to the active Triiodothyronine (T₃) within cells and peripheral tissues by deiodinases. As such, in contrast to Thyroxine (T₄), the Triiodothyronine (T₃) molecule contains three iodine atoms. Triiodothyronine (T₃) is the physiologically active thyroid hormone. It controls myocardium properties, heart rate, and vascular function. In fact, Triiodothyronine (T₃) affects almost every process in the body. Some suggest the thyroid gland produces T₃ directly. Although, thyroid disease is typically not treated with Triiodothyronine (T₃) supplementation. However, speculate a Thyroxine (T₄) and Triiodothyronine (T₃) combination might be better. Molecular structures are important because clinical effects resulting from thyroid hormone imbalance are observable at the cellular level.

As such, Thyroid hormone maintenance is extremely critical for adult metabolic activity, and thyroid hormone abnormalities in adolescence can have catastrophic consequences. Thyroid hormone imbalance can have profound effects on the central nervous system. Interestingly, Thyroid hormone receptors are located throughout the brain, highlighting their importance in central nervous system's development and function. Indeed, Triiodothyronine (T₃) and Thyroxine (T₄) also provide feedback to the brain and anterior pituitary gland to regulate thyroid hormone.

In adults, Hypothyroid symptoms include chronic fatigue, depression, impaired memory, and weight gain. In adolescents, Hypothyroid symptoms include poor growth trajectories, limited mental development, and delayed puberty. Additionally, hypothyroidism may cause problems such as sleep apnea, hypothermia, hypoventilation, neuropsychiatric syndromes, peripheral neuropathy, seizure, cerebellar ataxia, and coma.

Thyroid hormone replacement therapy may be a chronic and lifetime endeavor for treatment. Thyroid hormone dosage must be established for each patient individually. Usually, the initial dose is small, with amounts increasing gradually until clinical evaluation and laboratory tests indicate optimal response. The dose required to maintain this response is then continued. It is vital to patients physical and mental health that thyroid hormone treatment have the correct dosage. Under-treatment with thyroid hormone can have deleterious and disastrous consequences including neurochemical imbalances, depression, and fatigue.

Prior to the disclosed invention, the art reflected the idea that hypothyroidism could not be cured. Rather, since the year 1970s, the conventional wisdom has been that hypothyroidism is only treatable by replacing thyroid hormone deficiencies with orally administered levothyroxine (LT₄). And orally administered levothyroxine (LT₄) is still the standard treatment for hypothyroid patients despite its primitive conception five decades ago.

Another option for patients is T₃ or T₄ therapy options, such as thyroid armour. Thyroid armour is thyroid gland that has been dried and powdered for medical use. Typically, thyroid armour is made from animal thyroid glands. However, the most patients only use T₄ therapy. It remains unclear in the medical community whether combination therapy with T₃ or T₄ therapy is a better treatment option. In some cases, there may be worried that excessive T₃ could be associated with other health problems, such as arrhythmia. Ultimately, while T₄ is the predominant treatment option, each patient is different and requires careful analysis to get the best clinical results.

Now, artificial intelligence technologies are changing human health. For example, deep learning algorithms enable computer systems to have predictive power far superior to humans in various domains. Deep learning is a process by which machines learn using neural networks. Moreover, reinforcement learning algorithms provide an artificial intelligence structure for goal-oriented behavior. Typically, reinforcement learning programs have three parts. First the model defines an agent-environment relation. Second, a policy defines a decision-making process for the agent. Third, the reward defines the agent's goal, which is optimized according to the policy.

Despite advancements in other health disciplines, artificial intelligence has yet to have an impact for treating hypothyroidism. There are three major problems with daily oral levothyroxine for treating hypothyroidism: (1) Fluctuation in ingestion times and surrounding dietary conditions causes metabolic imbalance, (2) Fluctuation in patient needs causes metabolic imbalance, and (3) Fluctuation in brand name may cause metabolic imbalance. Thurs, there exists a need for new medical methods for patients to with hypothyroidism. The disclosure solves these three problems, providing methods for curing hypothyroidism with automatic monitoring and corrective release to maintain metabolic homeostasis.

### SUMMARY OF THE INVENTION

The invention is a process and system for curing hypothyroidism using artificial intelligence to measure blood and command hormonal secretion through a valve delivery mechanism. First, blood sensors measure hormonal concentration in the blood, storing measurements in memory for further processing the measurement. The sensors measure blood to detect and commit hormone levels to memory, including: Thyroxine (T₄), Triiodothyronine (T₃), Free Thyroxine (FT₄), and other thyroid hormones. Second, the measurements are processed and compared to target hormonal levels, and subsequently produce an automatic command for a valve control and drug delivery mechanism. Third, the valve control subsystem motivates thyroid hormone delivery from a supply of reserved hormone in a module to the patient's bloodstream optimizing hormonal homeostasis.

In certain embodiments, the present disclosure provides an implantable apparatus for automated thyroid hormone delivery control in a patient. The apparatus includes a hermetically sealed biocompatible housing configured for subcutaneous or intravascular implantation. A refillable reservoir disposed within the housing contains a pharmaceutical formulation of synthetic thyroid hormone. An integrated drug delivery system comprising a micropump, an electrically controllable microfluidic valve, and a delivery catheter allows precise release of the hormone directly into the patient's venous bloodstream. An implantable electrochemical biosensor continuously measures real-time concentrations of free thyroxine and free triiodothyronine in the bloodstream. An on-board controller housed within the apparatus processes the biosensor data using an artificial intelligence program to automatically regulate hormone delivery.

In certain embodiments, the artificial intelligence program includes at least one deep neural network that is trained on the patient's own historical physiological data. This deep neural network enables the system to calculate precise patient-specific dosage adjustments in real time. The controller accounts for natural circadian rhythm variations when determining the optimal therapeutic set-point for the patient. The on-board controller automatically actuates the micropump and microfluidic valve to deliver the calculated dosage through the catheter while minimizing overshoot. The apparatus maintains stable levels of free thyroxine and free triiodothyronine according to the patient's individual needs. This closed-loop system represents a significant improvement over conventional thyroid hormone replacement therapies that rely on periodic oral medication.

### BRIEF DESCRIPTION OF DRAWINGS

FIG. 1 illustrates embodiments of the disclosure as a drug delivery device.

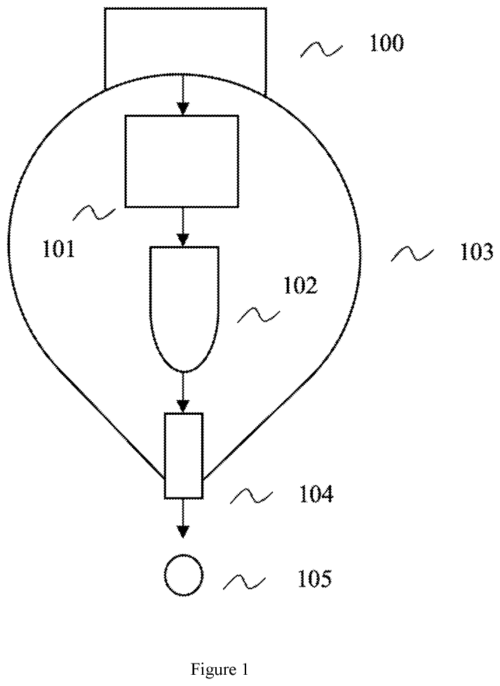

FIG. 2 illustrates methods for stabilizing and maintaining patient homeostasis.

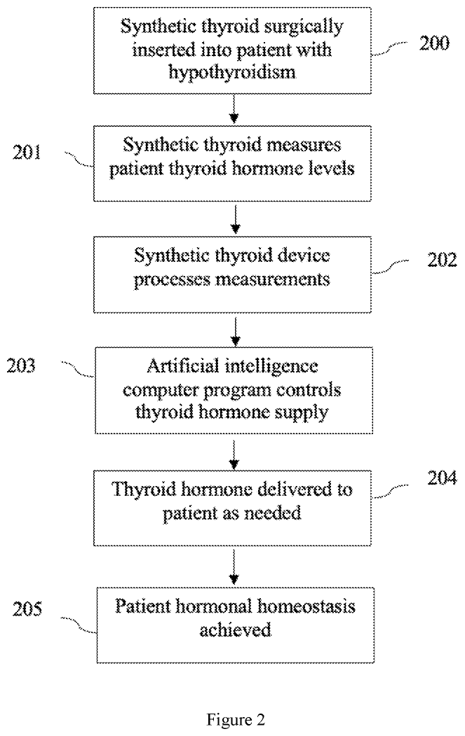

FIG. 3 illustrates embodiments of the disclosure as a process for intelligent metabolic manipulation in humans.

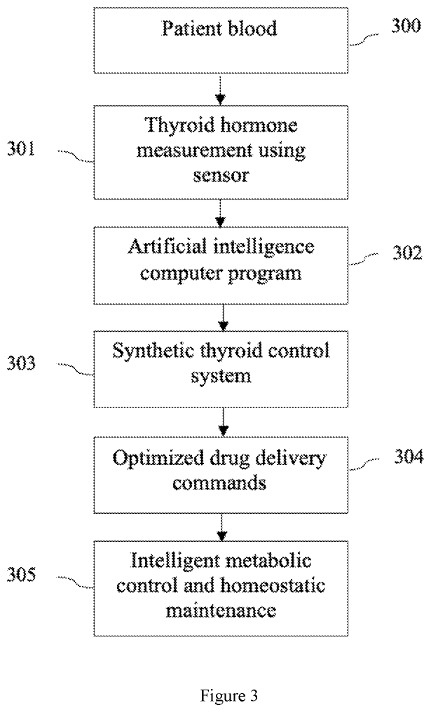

FIG. 4 illustrates embodiments of the disclosure as a synthetic thyroid.

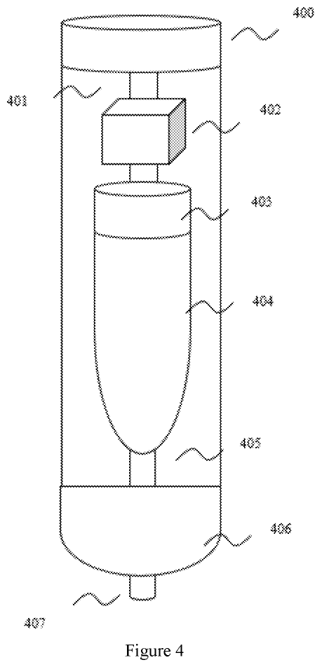

### DETAILED DESCRIPTION OF THE INVENTION

In certain embodiments, the disclosure is a drug delivery device 103 including a sensor 100, receiving information. The device processes the information with a micro-processor 101 and the processor generates instructions to command delivery of a thyroid hormone 102. The synthetic thyroid includes a delivery mechanism 104 which continuously delivers thyroid hormone to a patient's bloodstream 105.

In certain embodiments, the disclosure is methods for stabilizing and maintaining patient homeostasis where a synthetic thyroid is surgically inserted into the patient 200. The synthetic thyroid periodically measures the patient's hormone levels 201 and processes the measurements 202. The measurements are processed with a computer program 203 that produces instructions for thyroid hormone delivery 204, such that patient hormonal homeostasis is achieved 205.

In certain embodiments, the disclosure is a process where patient blood 300 flows to a sensor 301 for hormonal measurement using an artificial intelligence computer program 302 manipulating a Synthetic thyroid control system 303 and producing optimized drug delivery commands 304 for intelligent metabolic control and homeostatic maintenance 305.

In certain embodiments, the disclosure is a synthetic thyroid device. The device contains a sensor 400 that connects via wiring 401 to a microprocessor 402. The processor handles sensor data and sends signals to a pump 403 which creates pressure to move thyroxine from a reservoir 404 to a catheter 405 through a valve-controlled release chamber 406 and delivering the thyroxine to the patient's body 407.

In certain embodiments, the disclosure is a drug delivery device 103 including a sensor 100, receiving information and a micro-processor processing the information 101. The processor produces instructions to command delivery of a thyroid hormone from an embedded supply 102. Then, the instructions control a delivery mechanism 104 delivering thyroid hormone to a patient's bloodstream 105.

In certain embodiments, the disclosure is methods for stabilizing and maintaining patient homeostasis where a synthetic thyroid is surgically inserted into the patient 200. The synthetic thyroid periodically measures the patient's hormone levels 201 and processes the measurements 202 with an artificial intelligence computer program 203. In turn, the artificial intelligence computer program produces instructions for thyroid hormone delivery 204, such that patient hormonal homeostasis is achieved 205.

In certain embodiments, the disclosure is a process where patient blood 300 flows to a sensor 301 for hormonal measurement using an artificial intelligence computer program 302. The artificial intelligence computer program manipulates a Synthetic thyroid control system 303 and produces optimized drug delivery commands 304 for intelligent metabolic control and homeostatic maintenance 305.

In certain embodiments, the invention is a process by which a patient with hypothyroidism uses a synthetic thyroid 103 to supplement thyroid hormonal deficiencies, effectively curing the patient for as long as the synthetic thyroid provides the patient's needed hormone. In such embodiments, the disclosed invention provides a sustainable cure to hypothyroidism. In turn, the invention allows patients suffering from hypothyroidism to live without stresses associated medication management including daily reminders, neurochemical imbalances, and prescriptions. Thus, in certain patient's hypothyroid symptoms are effectively cured without the need for further treatment except for the regular replacement of the synthetic thyroid.

In certain embodiments, the invention is a process where a synthetic thyroid draws a blood sample. The blood sample is collected, using a measurement device, passing the measurement to a computer processor 101 using a convolutional neural network and reinforcement learning algorithm to command drug delivery from a Thyroxine (T₄) supply 102. The supply is loaded, to a release chamber and delivered to the bloodstream as a liquid drop. The delivery stabilizes hormonal homeostasis.

In certain embodiments, the present invention is a system for optimizing the hormone level of a patient wherein a data sensor 100 measures blood for hormonal concentration, and transfers a signal, to a processor with memory storage. Then, the hormonal measurement concentration is stored and processed, to subsequently deliver an open command, which is linked to a control valve via a computational coupling. This triggers thyroid hormone release, from a thyroid hormone store chamber containing thyroid hormone, to the bloodstream, by opening the control valve and subsequently commanding the control valve closed.

In certain embodiments, the synthetic thyroid uses a deep learning algorithm to control drug delivery 203 according to commands from a convolutional neural network. The information collected by blood measurements relating to thyroid hormone in the bloodstream may be processed with a neural network which predicts the needed dosing release. According to the prediction, the drug delivery system may deliver a specified amount of purified Thyroxine (T₄) to the bloodstream of the patient. In turn, the prediction ensures hormonal homeostasis for the patient over time by effectively linking the measurement and delivery mechanism in the synthetic thyroid via the neural network, which optimizes drug delivery.

In certain embodiments, the synthetic thyroid uses a deep learning algorithm to control drug delivery according to commands from an artificial intelligence computer program. The information collected by blood measurements relating to thyroid hormone in the bloodstream may be processed with an artificial intelligence 302 to predict the needed dosing release. According to the prediction, the drug delivery system delivers a specified amount of purified Thyroid Armor to the blood. In turn, the prediction promotes the stabilizing of hormonal homeostasis for the patient over time by effectively linking the measurement and delivery mechanism in the synthetic thyroid via the artificial intelligence computer program.

In certain embodiments, the drug delivery methods use a proximal policy optimization (PPO) algorithm, which is a type of reinforcement learning algorithm, to optimize drug delivery and homeostasis. In general, PPO works by computing an estimator of the policy gradient and iterating with a gradient optimization algorithm. In other words, the algorithm continuously updates the agent's policy based on the old policy's performance. Generally, the PPO update is a method of incremental improvement. In such embodiments, the proximal policy optimization algorithm may be trained in a simulation environment to develop a reinforcement learning agent. Then, the reinforcement learning agent is embedded to a processor inside the synthetic thyroid. In turn, the reinforcement learning agent sends commands to the drug delivery system according to an optimal decision-making algorithm 304, which promotes delivery to maintain metabolic homeostasis in the patient 205.

In certain embodiments, an artificial intelligence computer program 302 processes information relating to the patient's current thyroid hormone level. Then, the artificial intelligence program makes a prediction regarding the patient's needed thyroid hormone for maintaining homeostasis. Next, the prediction informs the amount of thyroid hormone pumped to a drug delivery chamber. Finally, the needed supply necessary for maintaining homeostasis is delivered to the patient's bloodstream 105.

In certain embodiments, the synthetic thyroid uses carbon fiber nanotubes for drug delivery. First, a sensor 100 measures thyroid hormones in the patient's bloodstream 301, which may include Thyroxine (T₄) and Free Thyroxine (FT₄) to identify patient need. Next, the sensor sends information to a drug delivery mechanism, which informs the measurement for thyroid hormone delivery. Then, the necessary thyroid hormone is delivered to the patient via carbon nanotubes, to ensure metabolic homeostasis 205.

In certain embodiments, the synthetic thyroid 100 is constructed with nanotechnologies including nanosensors, microprocessors, and carbon nanotubes. The synthetic thyroid may be constructed with multiple carbon nanotubes for performing two essential functions. The first function is measuring the blood for concentrations of Thyroxine (T₄) and Triiodothyronine (T₃). The second function is using the measured information to inform supply 102 delivery and deliver Thyroxine (T₄) to the bloodstream. Once delivered, the blood will perform deiodinases on the Thyroxine (T₄) generating more Triiodothyronine (T₃). The various functions may be performed using commands from a processor within the synthetic thyroid, communicating with nanotubes via nano-wiring.

In certain embodiments, the invention is a process and system for curing hypothyroidism using artificial intelligence. First, blood sensors measure thyroid hormone concentration in the blood of the patient. The sensors detect and commit hormone levels to memory, including: Thyroxine (T₄), Triiodothyronine (T₃), Free Thyroxine (FT₄), and other thyroid hormones. Second, the measurements are processed and compared to target hormonal levels, subsequently producing an automatic command for a valve control and delivery mechanism. The commands control the valve control and delivery mechanism such that the open command delivers thyroid hormone to the patient's bloodstream and the close command terminates the delivery process 304. Third, the valve control and drug delivery system command purified Thyroxine (T₄) delivery from a supply of reserved hormone in a module to the patient's bloodstream optimizing hormonal homeostasis 205.

In certain embodiments, the disclosure is a synthetic thyroid comprising a compact implantable housing measuring 25 mm in length, 15 mm in width, and 8 mm in height, fabricated from biocompatible titanium alloy. The device includes a micropump manufactured via micro-electromechanical systems technology with a piezoelectric actuator capable of delivering precise volumes at a rate of 0.1 to 10 microliters per minute, a solenoid valve with a 0.5 mm orifice diameter for flow control, and a reservoir holding 5 milliliters of concentrated thyroid hormone solution, levothyroxine at 100 micrograms per milliliter, sufficient for 3 months of continuous operation at average delivery rates.

In certain embodiments, the disclosure is a method for fabricating the synthetic thyroid where the titanium housing is precision-machined using computer numerical control (CNC) milling, followed by integration of the MEMS micropump and solenoid valve through laser welding. The reservoir is formed via additive manufacturing with polylactic acid (PLA) inner lining for drug stability, then filled with thyroid hormone under aseptic conditions, and the entire assembly is coated with a parylene polymer layer for enhanced biocompatibility before final sterilization via ethylene oxide gas.

In certain embodiments, the disclosure is a surgical implantation procedure for the synthetic thyroid where a 3 cm infraclavicular incision is made under local anesthesia, followed by creation of a subcutaneous pocket measuring 30 mm×20 mm×10 mm using blunt dissection. The device is inserted into the pocket with the delivery catheter, 1.5 mm outer diameter, 150 mm length, made of silicone tunneled subcutaneously to the left internal jugular vein, where a 2 mm venotomy is performed under ultrasound guidance. The catheter tip is positioned 20 mm above the right atrium, secured with a titanium anchor suture, and the device is affixed to the pectoral fascia using two non-absorbable sutures through pre-formed eyelets before layered closure and sterile dressing application.

In certain embodiments, the disclosure is a drug delivery device configured as a small implantable synthetic thyroid measuring 20 mm in length, 12 mm in width, and 6 mm in height, designed for insertion into the subcutaneous tissue of the patient's upper arm. The device incorporates an integrated micropump and solenoid valve system that delivers precise doses of thyroxine between 10 and 100 micrograms every 12 hours, with the pump utilizing a piezoelectric mechanism and the valve featuring a 0.3 mm orifice to ensure accurate bolus release from a 3-milliliter reservoir containing levothyroxine at 500 micrograms per milliliter concentration.

In certain embodiments, the disclosure is an implantation method for the synthetic thyroid where, under local anesthesia, a 2.5 cm incision is made on the medial aspect of the upper arm, followed by blunt dissection to create a subcutaneous pocket approximately 25 mm×15 mm×8 mm. The device is inserted into the pocket with its delivery catheter, 1.0 mm outer diameter, 50 mm length, silicone, positioned to release thyroxine directly into the local venous plexus, secured with a single non-absorbable suture through the device's fixation tab, and the incision closed with absorbable sutures and covered with a sterile occlusive dressing.

In certain embodiments, the disclosure is an operational sequence where the device's sensor monitors circulating thyroid hormone levels every 6 hours, processes data via an embedded microcontroller running a closed-loop algorithm, and activates the pump-valve system to deliver a calculated dose of 10 to 100 micrograms of thyroxine every 12 hours directly into the arm's venous return, thereby maintaining physiological hormone levels with minimal fluctuation and supporting long-term homeostatic control.

In certain embodiments, the disclosure is a drug delivery device configured as a small implantable synthetic thyroid, analogous in form factor to subdermal contraceptive implants, measuring 20 mm in length, 12 mm in width, and 6 mm in height, designed for insertion into the subcutaneous tissue of the patient's upper arm. Unlike polymer matrix systems that rely on passive diffusion and are prone to initial burst release, this device employs an active high-tech micropump and solenoid valve system that delivers precise doses of thyroxine between 10 and 100 micrograms every 12 hours, with the piezoelectric pump and 0.3 mm orifice valve ensuring controlled bolus release from a 3-milliliter reservoir containing levothyroxine at 500 micrograms per milliliter, thereby eliminating burst effects and enabling sustained, feedback-regulated hormone delivery.

In certain embodiments, the disclosure is an implantation method for the synthetic thyroid, performed similarly to contraceptive implant insertion, where under local anesthesia a 2.5 cm incision is made on the medial aspect of the upper arm, followed by use of a trocar to create a subcutaneous tunnel approximately 25 mm long. The device is inserted into the tunnel with its delivery catheter, 1.0 mm outer diameter, 50 mm length, silicone, positioned to release thyroxine directly into the local venous plexus, secured with a single non-absorbable suture through the device's fixation tab, and the incision closed with absorbable sutures and covered with a sterile occlusive dressing.

In certain embodiments, the disclosure is an operational sequence that addresses the burst release limitation of polymer matrix implants by utilizing active control, where the device's sensor monitors circulating thyroid hormone levels every 6 hours, processes data via an embedded microcontroller running a closed-loop algorithm, and activates the pump-valve system to deliver a calculated dose of 10 to 100 micrograms of thyroxine every 12 hours directly into the arm's venous return, thereby maintaining tight physiological control, preventing initial overdose, and supporting long-term homeostatic stability with programmable, on-demand adjustments.

In certain embodiments, the apparatus comprises an implantable closed-loop synthetic thyroid device having a hermetically sealed biocompatible housing configured for subcutaneous or intravascular implantation within a patient. The housing encloses a refillable reservoir, a micropump, an electrically controllable microfluidic valve, a delivery catheter, an electrochemical biosensor, and an on-board controller.

In certain embodiments, the apparatus includes a refillable reservoir disposed within the biocompatible housing, the reservoir containing a pharmaceutical formulation of synthetic thyroid hormone selected from the group consisting of levothyroxine (T4), liothyronine (T3), and combinations thereof, wherein the reservoir is designed to allow percutaneous refilling while maintaining sterility and long-term stability of the hormone formulation.

In certain embodiments, the apparatus further comprises an implantable electrochemical biosensor configured to continuously measure real-time concentrations of free T4 and free T3 directly in the patient's bloodstream, the biosensor being positioned on or integrated with the housing to provide high-frequency, accurate concentration data to the controller.

In certain embodiments, the apparatus includes an on-board controller integrated within the housing, the controller comprising a processor and non-transitory memory storing an artificial intelligence program that utilizes at least one deep neural network trained using patient-specific historical physiological data, the controller being operatively coupled to the biosensor, micropump, and microfluidic valve for closed-loop operation. In certain embodiments, the method comprises providing the implantable synthetic thyroid apparatus within the patient, continuously measuring real-time concentrations of free T4 and free T3 in the patient's bloodstream using the implanted electrochemical biosensor, and transmitting the measured concentration data to the on-board controller.

In certain embodiments, the method includes processing the measured free T4 and free T3 concentration data using an artificial intelligence computer program comprising at least one deep neural network to determine a precise dosage adjustment of thyroid hormone required by the patient.

In certain embodiments, the method further includes determining the dosage adjustment in real time while accounting for circadian rhythm variations and individual patient physiological patterns through the deep neural network trained on the patient's historical data to maintain a dynamic therapeutic set-point.

In certain embodiments, the method comprises automatically controlling the micropump and the electrically controllable microfluidic valve using the on-board controller to dispense the determined dosage of thyroid hormone directly into the patient's venous bloodstream via the delivery catheter in a manner that minimizes overshoot and maintains tight homeostatic control.

In certain embodiments, the method comprises executing an artificial intelligence software program on the on-board controller, wherein the software includes at least one deep neural network trained on the patient's historical thyroid hormone data, circadian patterns, and physiological responses to process real-time biosensor inputs, compute an optimized dosage adjustment, and generate corresponding control signals for the micropump and microfluidic valve.

In certain embodiments, the method further includes using the artificial intelligence software to implement a closed-loop feedback control routine that continuously evaluates the physiological response to each delivered dose, compares actual measured hormone levels against predicted values generated by the neural network, and dynamically refines the control parameters to minimize overshoot, reduce response time, and maintain stable thyroid hormone homeostasis over extended periods.

In certain embodiments, the implantable apparatus is used by first surgically implanting it subcutaneously in the patient's supraclavicular neck region under local anesthesia, with the catheter positioned to deliver into a suitable vein for direct bloodstream access. Once implanted, the device activates upon initialization via an external programmer that inputs patient-specific data such as age, weight, and comorbidities into the AI algorithm. The biosensor array continuously monitors FT4 and FT3 levels, feeding data to the microprocessor for real-time dosing adjustments, while the pump delivers microdoses as needed to maintain therapeutic thresholds. Healthcare providers monitor the system remotely through the encrypted Bluetooth module and companion app, receiving alerts for anomalies and performing over-the-air updates. Patients undergo periodic check-ups for reservoir refills via a percutaneous port, ensuring seamless long-term hormone management without daily oral medications.

In certain embodiments, the device's dosing process is governed by a pharmacokinetic model for hormone concentration, Equation 1.

$$C ( t ) = {C}_{0} {e}^{- kt} + \frac{D}{V} ( 1 - {e}^{- kt} ) \qquad ( 1 )$$

Here, C(t) is concentration at time t. C₀ is the initial concentration. k is the elimination rate, dis the delivered dose, and V is the volume of distribution.

In certain embodiments, the error calculation for feedback control, Equation 2.

e(t)=c_{target}−c_{measured}(t) (2)

Here, c_{target} is a predefined therapeutic threshold and c_{measured} is the real-time biosensor reading.

In certain embodiments, drug concentration may be predicted by Equation 3.

$${C}_{pred} ( t ) = C ( t ) + {\int }_{0}^{Δ t} ( - kC + \frac{D}{V} ) dt \qquad ( 3 )$$

Here, approximation is used for forward simulation, ensuring meticulous forecasting.

In certain embodiments, drug delivery may be defined by periodic impulse, Equation 4.

$$D ( t ) = {D}_{0} {\sum }_{n = 0}^{∞} δ ( t - n \cdot T ) \qquad ( 4 )$$

Here, D(t) is the instantaneous dose delivered at time (t).

In certain embodiments, the implantable apparatus operates through a sequential process beginning with biosensor measurement, where the integrated biosensor array continuously monitors thyroid hormone levels such as FT4 and FT3 in the bloodstream, along with auxiliary factors like pH and temperature for accurate readings. Following this, the collected data from the biosensor is transmitted to the embedded low-power microprocessor within the device's housing. The microprocessor then runs a neural network-based artificial intelligence algorithm to analyze the data, incorporating patient-specific factors such as age, weight, and comorbidities, and comparing against predefined therapeutic thresholds to determine if a dose adjustment is needed. If hormone levels are below target, the algorithm calculates the precise microdose required and signals the piezoelectric microfluidic pump to activate. Finally, the pump draws levothyroxine sodium from the reservoir and delivers the metered dose directly into the bloodstream via the extending catheter, completing the cycle with potential feedback for ongoing monitoring.

In certain embodiments, the software for controlling the apparatus comprises a pharmacokinetic simulation framework that models the dynamic absorption, distribution, metabolism, and elimination of synthetic thyroid hormone using ordinary differential equations solved via Euler integration.

In certain embodiments, the software executes real-time closed-loop control logic that processes biosensor inputs for free T4 and free T3 concentrations and generates actuator commands for the micropump and microfluidic valve to maintain target hormone levels.

In certain embodiments, the software includes a graphical user interface module that visualizes simulated hormone concentration curves over time, displays current delivery rates, and allows interactive adjustment of dosing parameters during development and testing.

In certain embodiments, the software incorporates patient-specific parameter tuning within the simulation environment to customize the pharmacokinetic model to individual metabolic rates, half-life values, and volume of distribution for more accurate predictive control.

In certain embodiments, the software is structured to support future integration of artificial intelligence modules by providing a modular observation-action-reward interface that can be directly coupled to reinforcement learning agents or neural networks for adaptive optimization of the delivery policy.

In certain embodiments, the synthetic thyroid incorporates a wireless telemetry module for real-time data transmission to an external programmer or companion mobile application. The module enables secure bidirectional communication using encrypted Bluetooth protocols to upload patient-specific historical data and lifestyle factors. The microprocessor 101 utilizes this incoming data to dynamically refine the parameters of the embedded deep neural network. Continuous model updates ensure the artificial intelligence program adapts to the patient's evolving metabolic profile over extended periods. This telemetry capability thereby supports remote physician oversight and over-the-air optimization of dosing algorithms without surgical intervention.

In certain embodiments, the apparatus includes multiple redundant safety mechanisms to prevent unintended over-delivery of thyroid hormone. A fail-safe solenoid valve automatically closes if the electrochemical biosensor detects hormone concentrations exceeding predefined upper thresholds. The on-board controller continuously monitors battery voltage, reservoir levels, and sensor integrity, triggering haptic or external alerts upon anomaly detection. Software-based cross-validation routines compare predicted versus measured hormone responses prior to every actuation of the micropump. These layered safeguards collectively minimize clinical risks and ensure reliable long-term operation of the closed-loop system.

In certain embodiments, the drug delivery system employs multi-sensor fusion techniques to enhance measurement accuracy beyond single-hormone readings. Auxiliary nanosensors integrated with the primary biosensor 100 capture complementary physiological parameters including core body temperature, heart rate variability, and blood pH levels. The artificial intelligence computer program 302 processes the fused dataset through a convolutional neural network architecture trained on patient-specific patterns. This integrated approach improves the precision of real-time dosing calculations under variable metabolic demands such as exercise or illness. Consequently, the synthetic thyroid achieves tighter homeostatic control and superior therapeutic consistency across diverse daily conditions.

In certain embodiments, the refillable reservoir is engineered for safe periodic percutaneous refilling using a dedicated external applicator device. The refill port features a self-sealing medical-grade septum that maintains sterility after repeated needle access under clinical supervision. Concentrated levothyroxine solution is injected directly into the reservoir while the microprocessor logs the exact volume and timestamps the event for compliance tracking. Predictive algorithms within the controller estimate remaining capacity based on historical delivery rates and alert the patient or physician when a refill is approaching. This design significantly extends the implant's service life and eliminates the need for frequent surgical replacement procedures.

In certain embodiments, the on-board controller executes a hybrid reinforcement learning framework that combines proximal policy optimization with supervised fine-tuning elements. The reinforcement learning agent is periodically retrained in a high-fidelity physiological simulation environment using anonymized multi-patient datasets while preserving individual privacy through federated learning techniques. Real-time biosensor feedback 100 drives continuous online policy updates to the synthetic thyroid control system 303. Simulation-validated improvements are deployed via secure over-the-air updates to optimize long-term metabolic homeostasis. Such advanced control strategies reduce dosage variability, minimize clinical interventions, and personalize therapy for each patient's unique physiological profile.

In certain embodiments, the synthetic thyroid device incorporates a long-life rechargeable power source consisting of a hermetically sealed lithium-ion micro-battery with a capacity sufficient for twelve months of continuous operation at nominal delivery rates. The battery is inductively charged through the skin using an external charger that communicates wirelessly with the on-board controller. A power-management module continuously monitors voltage levels and dynamically adjusts sensor sampling frequency and pump actuation to conserve energy while maintaining therapeutic accuracy. The artificial intelligence program 302 is programmed to enter a low-power sleep state between scheduled measurement cycles without compromising closed-loop control. This energy-efficient architecture thereby extends the functional lifespan of the implant and reduces the frequency of surgical interventions required for battery replacement.

In certain embodiments, the biocompatible housing of the synthetic thyroid is coated with a nanostructured titanium dioxide layer that promotes endothelial cell adhesion and minimizes chronic inflammatory response. The outer surface is further functionalized with anti-thrombogenic polyethylene glycol chains to prevent fibrin sheath formation around the delivery catheter. Long-term biocompatibility testing data is stored in the device memory for retrieval by the treating physician. The microprocessor 101 periodically logs tissue-impedance measurements to detect early signs of encapsulation or fibrosis. Such advanced surface engineering ensures the device remains functionally stable for years following implantation while preserving vascular access integrity.

In certain embodiments, the on-board controller includes a secure cryptographic module that supports encrypted over-the-air firmware updates and bidirectional communication with a cloud-based physician dashboard. Patient-specific thyroid hormone data, dosing history, and algorithm performance metrics are transmitted in anonymized form to a HIPAA-compliant server for aggregate analysis and model improvement. The artificial intelligence computer program 302 can receive remote parameter adjustments from an authorized clinician without requiring an office visit. Real-time alerts for out-of-range hormone levels or device anomalies are pushed to both the patient's smartphone application and the clinician's portal. This connected ecosystem enables proactive, data-driven optimization of therapy across large patient populations.

In certain embodiments, the synthetic thyroid supports combination therapy by maintaining separate reservoirs for levothyroxine (T4) and liothyronine (T3) that can be independently metered by the dual-channel microfluidic pump. The deep neural network is trained to calculate optimal T4/T3 ratios based on real-time free hormone levels, deiodinase activity estimates, and patient circadian rhythm data. Precise volumetric mixing occurs inside the release chamber immediately prior to delivery into the bloodstream. The controller continuously cross-validates the delivered ratio against measured post-delivery T3 and T4 concentrations to ensure pharmacokinetic fidelity. This dual-hormone capability provides physicians with greater flexibility to personalize treatment for patients who exhibit suboptimal response to T4 monotherapy.

In certain embodiments, the device firmware implements a multi-objective reinforcement learning policy that simultaneously optimizes hormone homeostasis, energy consumption, and patient comfort. The proximal policy optimization algorithm continuously evaluates trade-offs between dosing frequency, bolus size, and power draw using a patient-specific reward function updated via federated learning. Simulation-based stress testing confirms that the policy converges to stable control under simulated exercise, illness, and dietary perturbations. The resulting control policy is deployed to the embedded microprocessor 101 and refined online with every new biosensor reading. Consequently, the synthetic thyroid achieves superior long-term metabolic stability while minimizing device wear and patient-perceived interventions.

In certain embodiments, the synthetic thyroid employs a high-fidelity physiological simulation environment for offline training of the reinforcement learning agent prior to implantation. The simulation integrates patient-specific pharmacokinetic models, circadian rhythm profiles, and stochastic metabolic perturbations derived from historical biosensor data to generate millions of virtual dosing scenarios. The proximal policy optimization algorithm iteratively refines the control policy within this simulated environment until convergence criteria for hormone homeostasis and energy efficiency are satisfied. Once trained, the optimized policy is transferred to the on-board microprocessor 101 for real-time execution. This simulation-driven pre-training ensures robust closed-loop performance across a wide range of unencountered physiological conditions before the device ever interacts with the patient's bloodstream.

In certain embodiments, the embedded artificial intelligence computer program 302 utilizes a hybrid software architecture combining a convolutional neural network for real-time sensor data interpretation with a deep reinforcement learning controller for dosing decisions. The software continuously processes fused biosensor inputs through the neural network to predict future hormone trajectories over a rolling 24-hour horizon using the pharmacokinetic model of Equation 1. A model predictive control layer then evaluates thousands of candidate dosing sequences in a lightweight onboard simulator at each control cycle to select the optimal micro-bolus. The selected action is immediately executed by commanding the piezoelectric micropump and microfluidic valve while the software logs the outcome for subsequent policy improvement. This tightly integrated software-simulation loop enables predictive, proactive control that anticipates metabolic fluctuations rather than merely reacting to them.

In certain embodiments, the on-board controller implements continuous online simulation and policy adaptation using a digital twin of the patient's thyroid axis that runs in parallel with live operation. The digital twin is periodically synchronized with real-time biosensor readings 100 and recalibrated through federated learning updates received via the wireless telemetry module. The reinforcement learning agent compares actual measured hormone responses against predictions generated by the onboard simulator, computing policy gradients to incrementally improve the control policy without requiring full retraining. Safety bounds derived from extensive Monte-Carlo simulations constrain all online adaptations to guarantee that hormone levels remain within clinically validated therapeutic windows. This software-driven adaptive simulation framework allows the synthetic thyroid to personalize and refine its control strategy throughout the implant's lifetime, delivering increasingly precise metabolic homeostasis tailored to the individual patient.

In certain embodiments, the synthetic thyroid incorporates an integrated electrochemical biosensor positioned directly within the device housing to enable continuous sampling of the patient's bloodstream upon implantation. This biosensor is specifically engineered to detect free thyroxine (T4) levels through a competitive affinity-based immunoassay that relies on high-affinity monoclonal antibodies or synthetic aptamers immobilized on a gold electrode surface. The immunoassay operates by allowing free T4 molecules in the blood to compete with labeled T4 conjugates for binding sites, thereby producing a measurable change in electrical current or impedance that correlates directly with hormone concentration. The sensor is calibrated to achieve a detection limit of 0.1 ng/dL, ensuring reliable quantification even at sub-clinical thresholds where traditional assays may fail. Furthermore, the entire measurement cycle is optimized for a response time of less than 30 seconds, which supports rapid feedback loops within the closed-loop delivery system. Periodic self-calibration routines using internal reference standards maintain long-term accuracy and minimize drift over months of continuous operation.

In certain embodiments, the artificial intelligence computer program further utilizes a pharmacokinetic-pharmacodynamic (PK/PD) model of thyroid hormone dynamics that is personalized to the individual patient. This PK/PD model incorporates the patient's body mass index, age, any concurrent oral levothyroxine intake (if any), and diurnal variation patterns extracted from longitudinal sensor data and clinical records. The model processes these variables through compartmental differential equations to predict absorption, distribution, metabolism, and excretion rates, thereby generating a refined dosage value that accounts for real-time physiological fluctuations. This refined dosage output is subsequently supplied to the neural network, which translates it into precise control commands for the delivery pump and valve. The pump and valve are actuated to dispense thyroid hormone exclusively in discrete microgram increments, preventing overshoot and enabling fine-grained titration. The integrated control logic continuously enforces a personalized free T4 target range of 0.9-1.7 ng/dL, dynamically adjusting for circadian rhythms, physical activity, and other lifestyle factors to maintain euthyroid status and reduce adverse clinical events.

In certain embodiments, the implanted biosensor comprises an electrochemical affinity sensor having a gold working electrode surface that is functionalized with anti-free thyroxine (T4) monoclonal antibodies and a ferrocene redox mediator to enable highly selective and continuous detection of free T4 in whole blood. The functionalization process involves covalent attachment of the antibodies via a self-assembled monolayer of thiolated linkers, ensuring stable immobilization while preserving binding affinity over extended implantation periods. Upon exposure to the patient's bloodstream, free T4 molecules compete with the ferrocene-labeled T4 conjugate for antibody binding sites, resulting in a proportional change in the measured redox current that is processed by onboard electronics. The biosensor is calibrated in situ to achieve a limit of detection of 0.05 ng/dL, providing sensitivity well below typical clinical thresholds and allowing early detection of subtle hormonal fluctuations. Furthermore, the sensor architecture supports a response time of less than 20 seconds, facilitating true real-time feedback to the artificial intelligence control system without lag that could compromise homeostatic stability. Internal temperature compensation and anti-biofouling coatings maintain performance throughout the device's operational lifetime.

In certain embodiments, the at least one neural network is implemented as a convolutional long short-term memory network that is initially pre-trained on a large anonymized population dataset derived from thousands of thyroid disorder patients. Pre-training captures general patterns in thyroid hormone dynamics, circadian influences, and common comorbidities to establish robust baseline weights for the model. Following implantation, the network undergoes patient-specific fine-tuning using a minimum of six months of the individual's own historical data, including continuous free T4 readings, TSH levels, heart rate variability metrics, and accelerometer-derived physical activity profiles. This transfer-learning approach allows the convolutional long short-term memory network to adapt to the patient's unique metabolic signature, lifestyle factors, and response to micro-dosing while avoiding overfitting to population averages. The fine-tuned model processes incoming biosensor data in real time through convolutional layers that extract spatial-temporal features and long short-term memory network cells that retain long-term dependencies across days or weeks. Regular retraining cycles triggered by detected deviations further refine accuracy and ensure the neural network remains synchronized with evolving patient physiology.

In certain embodiments, the artificial intelligence computer program implements a patient-specific two-compartment pharmacokinetic-pharmacodynamic model that mathematically describes the absorption, distribution, metabolism, and excretion of both levothyroxine (T4) and triiodothyronine (T3). The model integrates the patient's age, body mass index, sex hormone levels, concurrent medications known to modulate deiodinase enzyme activity, and diurnal cortisol fluctuation data obtained from integrated sensors or external logs. Differential equations within the pharmacokinetic-pharmacodynamic framework are solved iteratively in real time to predict hormone concentrations and generate dosage recommendations exclusively in 0.5 microgram increments, thereby preventing overshoot and enabling precise titration. The refined dosage output is then passed to the neural network for final validation against the personalized homeostatic set-point range of 1.0-1.6 ng/dL. Safety constraints embedded in the model enforce upper and lower bounds on delivery rates and incorporate feedback from the biosensor to maintain euthyroid status across varying physiological states such as exercise, sleep, or illness.

In certain embodiments, the microfluidic delivery pathway comprises a titanium-coated silicon microchannel having an inner diameter of approximately 50 microns that connects the reservoir directly to the patient's bloodstream via a biocompatible cannula. The titanium coating enhances corrosion resistance and reduces protein adhesion, ensuring unobstructed flow over months of continuous operation. The artificial intelligence computer program precisely regulates the delivery pump and controllable valve to maintain a maximum flow rate of 2 microliters per minute, delivering hormone in controlled pulses that match the pharmacokinetic-pharmacodynamic model's output. A redundant occlusion sensor integrated into the microchannel continuously monitors backpressure, automatically triggering valve closure and an audible or wireless system alarm if backpressure exceeds 15 psi to prevent potential tissue damage or device malfunction. The entire control logic, including execution of the artificial intelligence computer program, is performed by a low-power processor housed within the implantable synthetic thyroid module, eliminating reliance on external computing devices and ensuring uninterrupted closed-loop operation even in the absence of wireless connectivity.

In certain embodiments, the at least one deep neural network is implemented as a convolutional long short-term memory deep neural network that is first pre-trained on population data collected from multiple thyroid disorder patients. Pre-training on this broad population data allows the convolutional long short-term memory deep neural network to learn general patterns in thyroid hormone regulation across diverse patient profiles. Following implantation, the convolutional long short-term memory deep neural network is subsequently fine-tuned using a minimum of six months of the specific patient's own historical free thyroxine levels, free triiodothyronine levels, heart rate variability data, and physical activity measurements. This fine-tuning process adapts the model to the individual patient's unique physiological responses and daily routines. The resulting patient-specific convolutional long short-term memory deep neural network enables highly accurate real-time dosage predictions that reflect the patient's personal metabolic characteristics over extended periods. Regular retraining cycles triggered by detected deviations from expected patterns further refine the convolutional long short-term memory deep neural network to maintain optimal performance throughout the life of the implanted apparatus.

In certain embodiments, the implantable electrochemical biosensor comprises a gold working electrode surface that is functionalized with monoclonal antibodies specific to free thyroxine and free triiodothyronine together with a ferrocene redox mediator. The functionalization process securely attaches the monoclonal antibodies and the ferrocene redox mediator to the gold surface through covalent bonding techniques to enable selective hormone binding and measurable signal generation. The biosensor is configured to continuously measure concentrations of free thyroxine and free triiodothyronine with a limit of detection of zero point zero five nanograms per deciliter in whole blood. This high sensitivity allows detection of even minor fluctuations in hormone levels before they become clinically significant. Additionally, the biosensor achieves a response time of less than twenty seconds, ensuring that concentration data is available to the controller without delay for immediate dosage adjustments. Anti-biofouling coatings applied to the electrode surface maintain long-term performance and prevent signal degradation during chronic implantation.

In certain embodiments, the on-board controller determines the precise patient-specific dosage adjustment exclusively in discrete increments of no greater than one-half microgram. The on-board controller maintains the dynamic therapeutic set-point for free thyroxine within a personalized range of one point zero to one point six nanograms per deciliter while simultaneously constraining free triiodothyronine levels according to the same real-time data. This incremental control strategy prevents sudden hormone spikes or drops that could destabilize the patient's physiological balance. The on-board controller continuously cross-validates the calculated dosage against both current biosensor readings and the patient-specific historical trends stored in memory. Safety limits embedded within the controller software enforce upper and lower bounds on each adjustment to guarantee that the delivered hormone remains within the personalized therapeutic window at all times.

In certain embodiments, the apparatus further comprises an integrated pressure sensor in fluid communication with the delivery catheter. The on-board controller continuously monitors backpressure through the integrated pressure sensor to detect any potential obstructions or flow anomalies in the delivery pathway. If the backpressure exceeds fifteen pounds per square inch, the on-board controller automatically closes the electrically controllable microfluidic valve to halt hormone delivery and prevent possible tissue damage. Simultaneously, the on-board controller activates an alarm that can be transmitted wirelessly to an external monitoring device or generate an audible alert perceptible to the patient. This redundant safety mechanism ensures that any occlusion or pressure buildup is addressed immediately while the apparatus remains fully implanted and operational.

In certain embodiments, the hermetically sealed biocompatible housing is constructed from titanium or a titanium alloy. The titanium or titanium alloy material provides exceptional corrosion resistance and mechanical strength while remaining lightweight enough for comfortable subcutaneous or intravascular implantation. Surface treatments applied to the titanium or titanium alloy housing promote tissue integration and minimize inflammatory responses over long-term use. The hermetic sealing process using laser welding techniques ensures that internal components remain protected from bodily fluids and maintain sterility throughout the device lifetime. This material choice also facilitates compatibility with magnetic resonance imaging procedures without causing image artifacts or device heating.

In certain embodiments, the refillable reservoir includes a self-sealing percutaneous refill port that allows replenishment of the pharmaceutical formulation of synthetic thyroid hormone while the apparatus remains implanted. The self-sealing percutaneous refill port is positioned on the outer surface of the hermetically sealed biocompatible housing and is accessible using a standard non-coring needle under local anesthesia. A septum made from medical-grade silicone within the refill port reseals automatically after needle withdrawal to prevent leakage or contamination. This design enables periodic refilling of the reservoir without requiring surgical removal of the apparatus. The refill procedure can be performed in an outpatient setting and includes verification steps to confirm proper volume restoration and system integrity before resuming normal operation.

In certain embodiments, the artificial intelligence program is further configured to modify the dynamic therapeutic set-point over time based on detected changes in the patient's physiological patterns. The artificial intelligence program analyzes long-term trends in free thyroxine and free triiodothyronine concentrations along with correlated physiological signals to identify evolving needs such as those caused by aging or lifestyle shifts. Adjustments to the dynamic therapeutic set-point are made gradually and only after confirmation across multiple measurement cycles to avoid transient errors. The modified set-point is stored in the non-transitory memory of the on-board controller and becomes the new reference for all subsequent dosage calculations. This adaptive capability ensures that the apparatus remains aligned with the patient's changing physiology without requiring manual reprogramming by a clinician.

In certain embodiments, the artificial intelligence program stored in the on-board controller processes both current real-time concentration data from the biosensor and previously accumulated patient-specific historical physiological data to predict upcoming changes in hormone requirements. The artificial intelligence program employs predictive algorithms to forecast short-term and medium-term fluctuations in free thyroxine and free triiodothyronine needs based on circadian patterns, activity levels, and historical responses. Proactive dosage adjustments are calculated and implemented before deviations from the dynamic therapeutic set-point actually occur. The on-board controller logs each predictive adjustment along with the corresponding biosensor outcomes to continuously improve the accuracy of future predictions. This forward-looking control strategy minimizes the time the patient spends outside the optimal hormone range and enhances overall therapeutic consistency.

In certain embodiments, a system is governed by the following ordinary differential equations, where Equation 5 defines an absorbing pool.

$$\frac{dA}{dt} = - {k}_{a} A \qquad ( 5 )$$

Equation 6 defines a serum free T4 pool.

$$\frac{dF}{dt} = {k}_{a} A \cdot s - {k}_{e} F \qquad ( 6 )$$

The rate constants are defined from half-lives, as defined by Equation 7.

$${k}_{a} = \frac{ln 2}{{t}_{1 / 2 , absorb}} , {k}_{e} = \frac{ln 2}{{t}_{1 / 2 , elim}} \qquad ( 7 )$$

The differential equations are solved numerically using the forward Euler method with a small, fixed time step. At each dosing event the absorbing pool is instantaneously increased by the administered dose. In such embodiments, the simulation runs for a user-specified duration and supports interactive adjustment of dose, dosing interval, and visualization speed via the GUI.

It is to be understood that while certain embodiments and examples of the invention are illustrated herein, the invention is not limited to the specific embodiments or forms described and set forth herein. It will be apparent to those skilled in the art that various changes and substitutions may be made without departing from the scope or spirit of the invention and the invention is not considered to be limited to what is shown and described in the specification and the embodiments and examples that are set forth therein. Moreover, several details describing structures and processes that are well-known to those skilled in the art and often associated with biotechnologies are not set forth in the following description to better focus on the various embodiments and novel features of the disclosure of the present invention. One skilled in the art would readily appreciate that such structures and processes are at least inherently in the invention and in the specific embodiments and examples set forth herein.

One skilled in the art will readily appreciate that the present invention is well adapted to carry out the objectives and obtain the ends and advantages mentioned herein as well as those that are inherent in the invention and in the specific embodiments and examples set forth herein. The embodiments, examples, methods, and compositions described or set forth herein are representative of certain preferred embodiments and are intended to be exemplary and not limitations on the scope of the invention. Those skilled in the art will understand that changes to the embodiments, examples, methods and uses set forth herein may be made that will still be encompassed within the scope and spirit of the invention. Indeed, various embodiments and modifications of the described compositions and methods herein which are obvious to those skilled in the art, are intended to be within the scope of the invention disclosed herein. Moreover, although the embodiments of the present invention are described in reference to use in connection with artificial intelligence, ones of ordinary skill in the art will understand that the principles of the present inventions could be applied to other types of biotechnology for a wide variety of applications.

I claim:

1. A method for thyroid hormone delivery control, the method comprising:
providing a synthetic thyroid, the synthetic thyroid containing one drug delivery system, with a delivery pump, a valve, and a reservoir containing thyroid hormone;
measuring a concentration of thyroid hormone in the patient's bloodstream;
using an artificial intelligence computer program to determine a dosage of thyroid hormone based on the measured concentration;
wherein the artificial intelligence computer program uses at least one neural network; and
controlling the delivery pump and the valve using the artificial intelligence computer program to dispense the dosage of thyroid hormone into the patient's bloodstream.

2. The method of claim 1, wherein artificial intelligence computer program used to determine a dosage of thyroid hormone based on the measured concentration further comprises at least one expert system.

3. The method of claim 1, wherein artificial intelligence computer program used to determine a dosage of thyroid hormone based on the measured concentration further comprises at least one reinforcement learning program.

4. The method of claim 1, wherein the synthetic thyroid is configured as an implantable medical device.

5. The method of claim 1, wherein the artificial intelligence computer program further utilizes a pharmacokinetic-pharmacodynamic model of thyroid hormone dynamics that incorporates the patient's body mass index, age, concurrent oral levothyroxine intake, and diurnal variation patterns to generate a refined dosage value that is then used by the neural network to control the delivery pump and valve in discrete microgram increments while enforcing a personalized free T4 target range of 0.9-1.7 ng/dL.

6. The method of claim 1, wherein the concentration of thyroid hormone in the patient's bloodstream is measured by an integrated electrochemical biosensor located within the synthetic thyroid that specifically detects free thyroxine (T4) levels using a competitive affinity-based immunoassay with a detection limit of 0.1 ng/dL and a response time of less than 30 seconds.

7. A method for thyroid hormone delivery control, the method comprising:
providing an implantable synthetic thyroid module containing a drug delivery system, the drug delivery system comprising a delivery pump, a controllable valve, and a biocompatible reservoir holding a supply of synthetic thyroid hormone;
continuously measuring, with an implanted biosensor, a concentration of free thyroid hormone in the patient's bloodstream;
using an artificial intelligence computer program comprising at least one neural network to process the measured concentration in real time and determine a precise dosage adjustment of thyroid hormone needed to maintain a patient-specific homeostatic set-point; and
automatically controlling the delivery pump and valve with the artificial intelligence computer program to dispense the determined dosage directly into the patient's bloodstream via a microfluidic delivery pathway.

8. The method of claim 7, wherein the implanted biosensor comprises an electrochemical affinity sensor having a gold working electrode surface functionalized with anti-free thyroxine (T4) monoclonal antibodies and a ferrocene redox mediator, the biosensor being configured to continuously and selectively detect free T4 concentration with a limit of detection of 0.05 ng/dL and a response time of less than 20 seconds in whole blood.

9. The method of claim 7, wherein the at least one neural network is a convolutional long short-term memory network that is first pre-trained on a large population dataset of thyroid disorder patients and is subsequently fine-tuned in a patient-specific manner using a minimum of six months of the patient's own historical free T4, TSH, heart rate variability, and accelerometer-derived activity data.

10. The method of claim 7, wherein the microfluidic delivery pathway includes a titanium-coated silicon microchannel with an inner diameter of approximately 50 microns, and wherein the artificial intelligence computer program controls the pump and valve assembly to deliver thyroid hormone at a controlled maximum flow rate of 2 microliters per minute while continuously monitoring backpressure via a redundant occlusion sensor that triggers automatic valve closure and system alarm if backpressure exceeds 15 psi.

11. The method of claim 7, wherein the artificial intelligence computer program is executed by a processor housed within the implantable synthetic thyroid module.

12. An apparatus for thyroid hormone delivery control, comprising:
a hermetically sealed biocompatible housing configured for subcutaneous or intravascular implantation in the patient;
a refillable reservoir disposed within the housing and containing a pharmaceutical formulation of synthetic thyroid hormone selected from the group consisting of levothyroxine T4, liothyronine T3, and combinations thereof;
a drug delivery system contained within the housing, the drug delivery system comprising a micropump, an electrically controllable microfluidic valve, and a delivery catheter configured to release the hormone directly into the patient's venous bloodstream;
an implantable electrochemical biosensor positioned to continuously measure real-time concentrations of free T4 and free T3 in the patient's bloodstream; and
an on-board controller integrated within the biocompatible housing and operatively coupled to the biosensor, the micropump, and the valve, the controller comprising a processor and non-transitory memory storing an artificial intelligence program that includes at least one deep neural network trained on patient-specific historical physiological data;
wherein the controller is configured to:
receive the real-time free T4 and free T3 concentration data from the biosensor, determine a precise patient-specific dosage adjustment in real time using the deep neural network to maintain a dynamic therapeutic set-point that accounts for circadian rhythm variations, and
automatically actuate the micropump and microfluidic valve to deliver the dosage adjustment directly into the patient's bloodstream via the catheter while minimizing overshoot.

13. The apparatus of claim 12, wherein the at least one deep neural network is a convolutional long short-term memory deep neural network that is first pre-trained on population data from multiple thyroid disorder patients and is subsequently fine-tuned using a minimum of six months of the specific patient's own historical free thyroxine levels, free triiodothyronine levels, heart rate variability data, and physical activity measurements.

14. The apparatus of claim 12, wherein the implantable electrochemical biosensor comprises a gold working electrode surface functionalized with monoclonal antibodies specific to free thyroxine and free triiodothyronine together with a ferrocene redox mediator, the biosensor being configured to continuously measure concentrations of free thyroxine and free triiodothyronine with a limit of detection of 0.05 nanograms per deciliter and a response time of less than twenty seconds in whole blood.

15. The apparatus of claim 12, wherein the on-board controller determines the precise patient-specific dosage adjustment exclusively in discrete increments of no greater than one-half microgram and maintains the dynamic therapeutic set-point for free thyroxine within a personalized range of 1.0 to 1.6 nanograms per deciliter while simultaneously constraining free triiodothyronine levels according to the same real-time data.

16. The apparatus of claim 12, further comprising an integrated pressure sensor in fluid communication with the delivery catheter, wherein the on-board controller continuously monitors backpressure and automatically closes the electrically controllable microfluidic valve and activates an alarm if the backpressure exceeds fifteen pounds per square inch.

17. The apparatus of claim 12, wherein the hermetically sealed biocompatible housing is constructed from titanium or a titanium alloy.

18. The apparatus of claim 12, wherein the refillable reservoir includes a self-sealing percutaneous refill port that allows replenishment of the pharmaceutical formulation of synthetic thyroid hormone while the apparatus remains implanted.

19. The apparatus of claim 12, wherein the artificial intelligence program is further configured to modify the dynamic therapeutic set-point over time based on detected changes in the patient's physiological patterns.

20. The apparatus of claim 12, wherein the artificial intelligence program stored in the on-board controller processes both current real-time concentration data from the biosensor and previously accumulated patient-specific historical physiological data to predict upcoming changes in hormone requirements and to proactively adjust the delivered dosage of synthetic thyroid hormone.
